(19) United States
(12) Patent Application Publication
(10) Pub. No.: US 2023/0329753 A1
(43) Pub. Date: Oct. 19, 2023

(54) INTRAVENOUS ARTERIAL COMPLIANCE RESTORATION

(71) Applicant: EDWARDS LIFESCIENCES CORPORATION, Irvine, CA (US)

(72) Inventor: Arvin T. Chang, Yorba Linda, CA (US)

(21) Appl. No.: 18/336,321

(22) Filed: Jun. 16, 2023

Related U.S. Application Data
(63) Continuation of application No. PCT/US21/63765, filed on Dec. 16, 2021.
(60) Provisional application No. 63/199,324, filed on Dec. 18, 2020.

(51) Int. Cl. A61B 17/34; A61B 17/11

(52) U.S. Cl. CPC A61B 17/3468; A61B 17/11; A61B 2017/1107; A61B 2017/1139

(57) ABSTRACT

A method of shunting blood involves forming a first opening in a wall of a first blood vessel and a wall of a second blood vessel, anchoring a first port of a compliant fluid container to the wall of the first blood vessel such that the first port provides access between the first blood vessel and the second blood vessel through the first opening, and placing a body of the compliant fluid container within the second blood vessel.

### RELATED APPLICATION

This application is a continuation of International Patent Application No. PCT/US2021/063765, filed Dec. 16, 2021, which claims the benefit of U.S. Patent Application No. 63/199,324, filed Dec. 18, 2020, the entire disclosures of each of which are incorporated by reference for all purposes.

### BACKGROUND

The present disclosure generally relates to the field of medical implant devices.

### DESCRIPTION OF RELATED ART

Insufficient or reduced compliance in certain blood vessels, including arteries such as the aorta, can result in reduced perfusion, cardiac output, and other health complications. Restoring compliance to such blood vessels can improve patient outcomes.

### SUMMARY

Described herein are devices, methods, and systems that facilitate the restoration of compliance characteristics to undesirably stiff blood vessels. Devices associated with the various embodiments of the present disclosure can include compliant body features configured to be positioned/disposed within a venous blood vessel when the device is implanted in fluid communication with an arterial blood vessel to increase compliance thereof.

In some implementations, the present disclosure relates to a method of shunting blood. The method comprises forming a first opening in a wall of a first blood vessel and a wall of a second blood vessel, anchoring a first port of a compliant fluid container to the wall of the first blood vessel such that the first port provides access between the first blood vessel and the second blood vessel through the first opening, and placing a body of the compliant fluid container within the second blood vessel.

In some embodiments, the first blood vessel is an artery and the second blood vessel is a vein.

The method can further comprise channeling blood from the first blood vessel into the body of the compliant fluid container within the second blood vessel through the first port. For example, in some implementations, the method further comprises forming a second opening in the wall of the first blood vessel and the wall of the second blood vessel, anchoring a second port of the compliant fluid container to the wall of the first blood vessel such that the second port provides access between the first blood vessel and the second blood vessel through the second opening, and channeling blood from the body of the compliant fluid container into the first blood vessel through the second port.

The method can further comprise passing blood through the body of the compliant fluid container between the first port and the second port. In some implementations, the first port is upstream of the second port with respect to blood flow within the first blood vessel.

In some implementations, the method further comprises forming a third opening in the wall of the first blood vessel and the wall of the second blood vessel, anchoring a third port of the compliant fluid container to the wall of the first blood vessel such that the third port provides access between the first blood vessel and the second blood vessel through the second opening, and channeling blood between the first blood vessel and the body of the compliant fluid container through the third port.

The method can further comprise forming a second opening in a wall of a third blood vessel and a wall of a fourth blood vessel, anchoring a second port of the compliant fluid container to the wall of the third blood vessel such that the second port provides access between the third blood vessel and the second blood vessel through the second opening, and channeling blood from the body of the compliant fluid container into the third blood vessel through the second port.

A portion of the body of the compliant fluid container may be disposed within the fourth blood vessel. In some embodiments, the first blood vessel is an aorta, the second blood vessel is inferior vena cava, the third blood vessel is an iliac artery, and the fourth blood vessel is an iliac vein.

In some embodiments, the first port is formed by an anchoring structure of the compliant fluid container that is disposed within the first opening. For example, the anchoring structure can comprise a stent configured to hold open the first opening.

The method may further comprise adding compliance to the first blood vessel by filling the body of the compliant fluid container with blood from the first blood vessel to thereby expand the body of the compliant fluid container within the second blood vessel.

In some implementations, the present disclosure relates to a compliance restoration implant device comprising a compliant fluid container configured such that a cross-sectional area of the fluid container increases when a pressure level within the fluid container is greater than a pressure level outside of the fluid container and decreases when the pressure level within the fluid container is less than the pressure level outside of the fluid container, and a first port structure coupled to the fluid container and configured to provide fluid access to an interior of the fluid container.

The first port structure can be configured to be anchored to a blood vessel wall.

In some embodiments, the first port structure comprises a stent frame.

The compliance restoration implant device can further comprise a second port structure coupled to the fluid container and configured to provide fluid access to the interior of the fluid container. For example, in some embodiments, the first port structure is coupled to a first end of the fluid container and the second port structure is coupled to a second end of the fluid container.

The compliance restoration implant device can further comprise a third port structure coupled to the fluid container and configured to provide fluid access to the interior of the fluid container. In some embodiments, the first port structure has an opening that is greater than an opening of the second port structure.

In some embodiments, the fluid container comprises a tubular member and a sleeve disposed about the tubular member. For example, the sleeve can be configured such that a cross-section thereof changes from an oval shape to a more circular shape in response to an increase in pressure within the tubular member. In some embodiments, the sleeve is elastic. In some embodiments, the sleeve comprises a memory metal frame. In some embodiments, the sleeve comprises a braided mesh.

In some implementations, the present disclosure relates to a fluid bypass implant device comprising a compliant tubular structure, a first fluid port associated with a first end of the tubular structure, and a second fluid port associated with a second end of the tubular structure.

Each of the first fluid port and the second fluid port can comprise an anchoring means configured to anchor to an interior wall of a blood vessel. For example, the anchoring means comprises one or more anchoring arms that extend from a respective one of the first fluid port and the second fluid port and are configured to contact the interior wall of the blood vessel. In some embodiments, the anchoring means comprises a flange structure.

In some embodiments, the fluid bypass implant device further comprises a flow control means disposed at least partially within a fluid channel of the fluid bypass implant device. For example, the flow control means can comprise a one-way valve.

The fluid bypass implant device may comprise one or more valve devices coupled respectively to one or more of the first fluid port and the second fluid port.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example representation of a heart and associated vasculature having various features relevant to one or more embodiments of the present inventive disclosure.

FIGS. 2A and 2B provide cross-sectional and side views, respectively, of a blood vessel experiencing compliant expansion during the systolic phase of the cardiac cycle.

FIGS. 3A and 3B provide cross-sectional and side views, respectively, of the artery shown in FIGS. 2A and 2B during the diastolic phase of the cardiac cycle.

FIG. 4 is a graph illustrating blood pressure over time in an example healthy patient.

FIG. 5 is a graph illustrating blood pressure over time in an example patient having reduced aortic compliance.

FIG. 6 is a cross-sectional view of a compliance-restoration device implanted in arterial and venous blood vessels in accordance with one or more embodiments.

FIG. 7 shows a side view of a compliance-restoration device including stent-type port reinforcement structures in accordance with one or more embodiments.

FIG. 8A shows a side view of a compliance-restoration device including a compliant sleeve associated with a body portion thereof in accordance with one or more embodiments.

FIGS. 8B and 8C show cross-sectional views of the compliant sleeve of FIG. 8A in compressed and expanded configurations, respectively, in accordance with one or more embodiments.

FIG. 9 is a cross-sectional view of a compliance-restoration device including port structures having differing geometries in accordance with one or more embodiments.

FIG. 10 is a cross-sectional view of a compliance-restoration device including flow-control features in accordance with one or more embodiments.

FIG. 11 is a cross-sectional view of a compliance-restoration device including more than two ports in accordance with one or more embodiments.

FIG. 12 is a cross-sectional view of a compliance-restoration device implanted in arterial and venous blood vessels in accordance with one or more embodiments.

FIG. 13 is a cross-sectional view of a single-port compliance-restoration device implanted in arterial and venous blood vessels in accordance with one or more embodiments.

FIGS. 14-1, 14-2, 14-3, 14-4, and 14-5 illustrate a flow diagram for a process for implanting a compliance restoration device in accordance with one or more embodiments.

FIGS. 15-1, 15-2, 15-3, 15-4, and 15-5 provides images of the compliance restoration device and certain anatomy corresponding to operations of the process of FIGS. 14-1, 14-2, 14-3, 14-4, and 14-5 according to one or more embodiments.

### DETAILED DESCRIPTION

The headings provided herein are for convenience only and do not necessarily affect the scope or meaning of the claimed invention.

Although certain preferred embodiments and examples are disclosed below, it should be understood that the inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Compliance and Anatomy

Certain embodiments are disclosed herein in the context of vascular implant devices, and in particular, compliance-restoration implant devices implanted in the aorta and/or inferior vena cava. However, although certain principles disclosed herein may be particularly applicable to the anatomy of the aorta and inferior vena cava, it should be understood that compliance-restoration implant devices in accordance with the present disclosure may be implanted in, or configured for implantation in, any suitable or desirable blood vessels or other anatomy.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart, which is discussed in detail below.

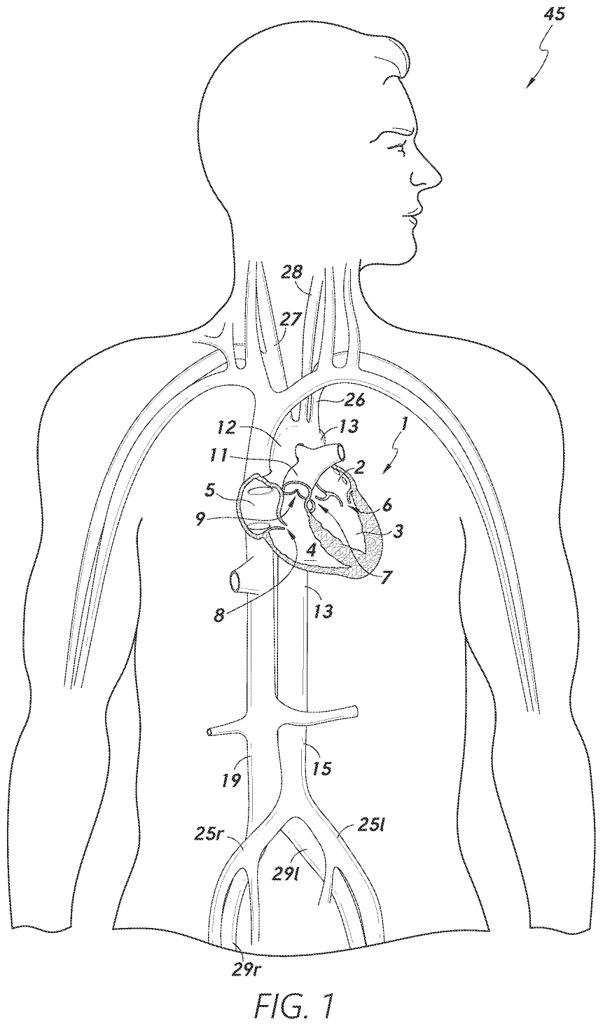

FIG. 1 illustrates an example representation of a heart 1 and associated vasculature having various features relevant to one or more embodiments of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11.

The pulmonary artery 11 carries deoxygenated blood from the right side of the heart to the lungs. The pulmonary artery 11 includes a pulmonary trunk and left and right pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Both arteries and veins are types of blood vessels in the cardiovascular system. Generally, arteries, such as the aorta, carry blood away from the heart, whereas veins, such as the inferior and superior venae cavae, carry blood back to the heart.

As mentioned above, the aorta is coupled to the heart 1 via the aortic valve 7, which leads into the ascending aorta 12 and gives rise to the innominate artery 27, the left common carotid artery 28, and the left subclavian artery 26 along the aortic arch before continuing as the descending thoracic aorta 13 and further the abdominal aorta 15. References herein to the aorta may be understood to refer to the ascending aorta (also referred to as the “ascending thoracic aorta”), aortic arch, descending aorta, thoracic aorta (also referred to as the “descending thoracic aorta”), abdominal aorta, or other arterial blood vessel or portion thereof.

Arteries, such as the abdominal aorta 15, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, or the tendency of a blood vessel (e.g., artery) or prosthetic implant device, or portion thereof, to resist recoil toward its original dimensions on application of a distending or compressing force. Compliance of a blood vessel or prosthetic implant device may or may not be based on elasticity or stretchability of the blood vessel walls.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

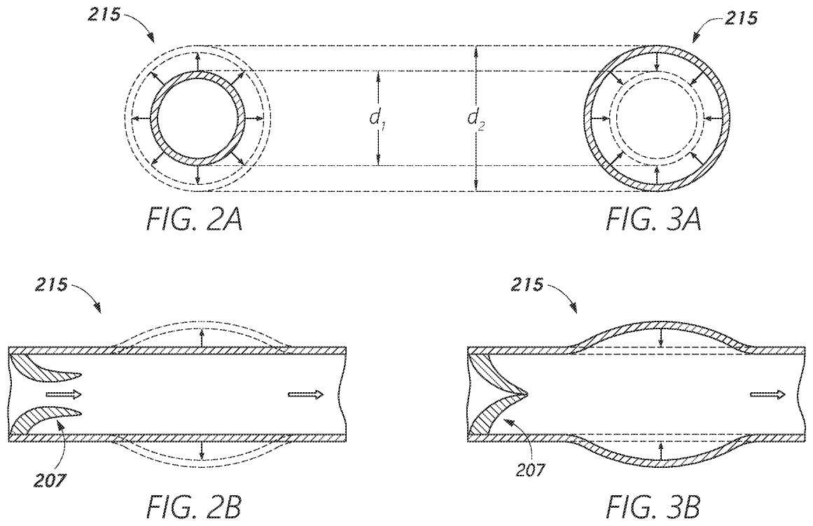

FIGS. 2A and 2B provide cross-sectional and side views, respectively, of a blood vessel 215, such as an artery (e.g., aorta), experiencing expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume will generally occur in an artery when the pressure in the artery is increased. With respect to the aorta, as shown in FIGS. 2A and 2B, as blood is pumped into the aorta 215 through the aortic valve 207, the pressure in the aorta increases and the diameter of at least a portion of the aorta expands. A first portion of the blood entering the aorta 215 during systole may pass through the aorta during the systolic phase, while a second portion (e.g., approximately half of the total blood volume) may be stored in the expanded volume caused by arterial compliance, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure and/or volume conditions. Compliance (C) may be calculated using the following equation, where ΔV is the change in volume (e.g., in mL), and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the thoracic and/or abdominal aorta. However, it should be understood that such devices, methods and/or concepts may be applicable in connection with any other artery or blood vessel.

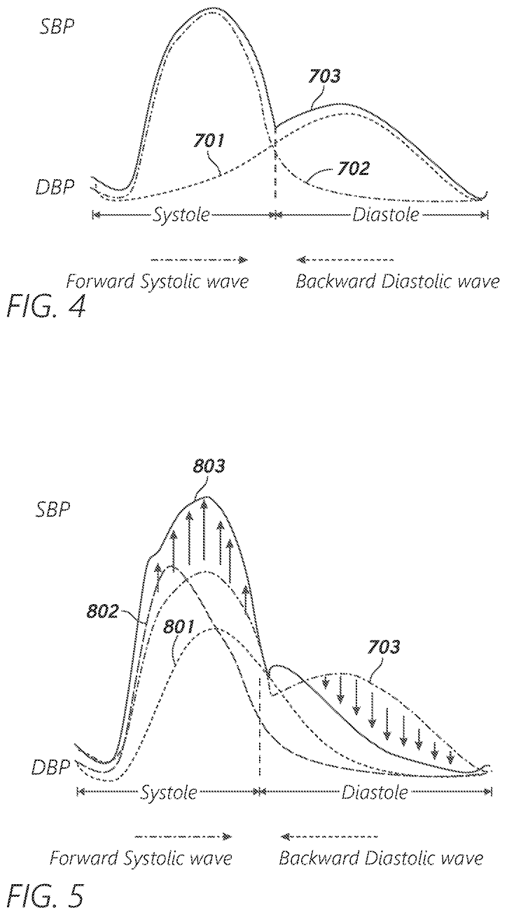

FIGS. 3A and 3B provide cross-sectional and side views, respectively, of the artery 215 shown in FIGS. 2A and 2B during the diastolic phase of the cardiac cycle. As shown, arterial compliance may cause retraction of the blood vessel wall inward during diastole, thereby creating pressure to continue to push blood through the artery 215 when the valve 207 is closed. For example, during systole, approximately 50% of the blood that enters the artery 215 through the valve 207 may be passed through the artery, whereas the remaining 50% may be stored in the artery, as enabled by expansion of the vessel wall. Some or all of the stored portion of blood in the artery 215 may be pushed through the artery by the contracting vessel wall during diastole. For patients experiencing arterial stiffness (i.e., lack of compliance), their arteries may not operate effectively in accordance with the expansion/contraction functionality shown in FIGS. 2A and 2B and FIGS. 3A and 3B.

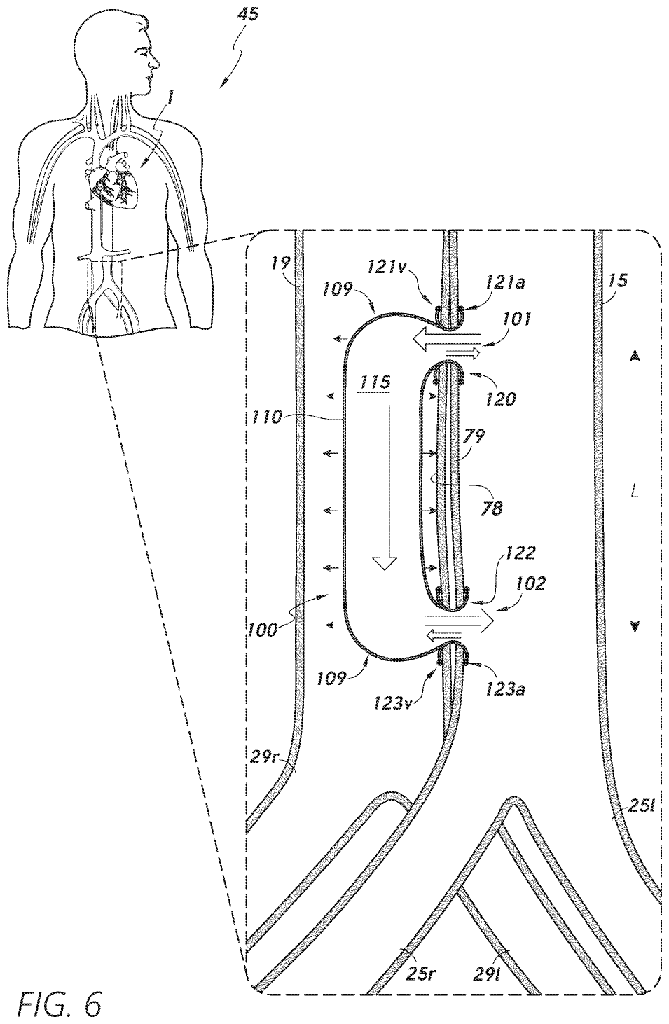

FIG. 4 is a graph illustrating blood pressure over time in an example healthy patient, wherein arterial blood pressure is represented as a combination of a forward systolic pressure wave 701 and a backward diastolic pressure wave 702. The combination of the systolic wave 701 and the diastolic wave 702 are represented by the waveform 703.

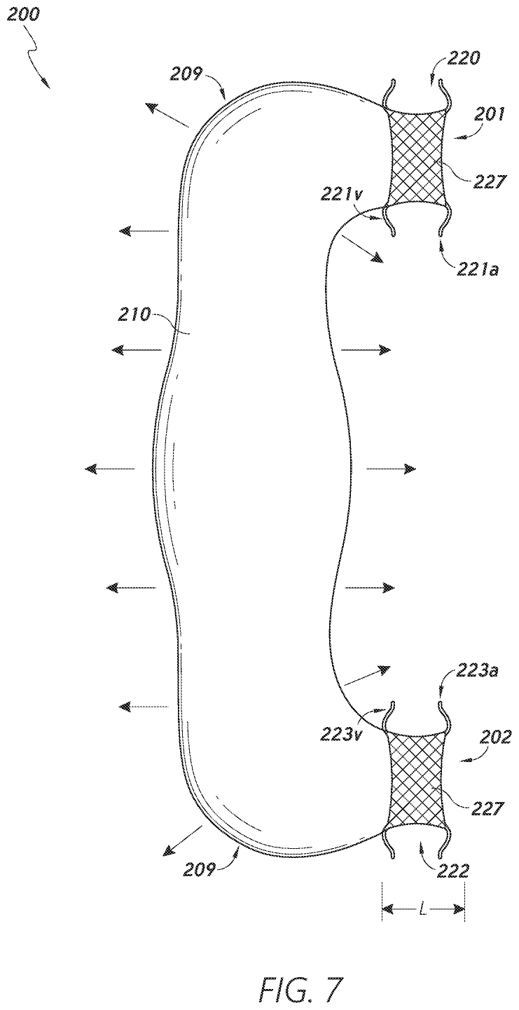

FIG. 5 is a graph illustrating blood pressure over time in an example patient having reduced aortic compliance. The graph of FIG. 5 shows, for reference purposes, the example combined wave 703 shown in FIG. 4. When low compliance is exhibited, less energy may be stored in the aorta compared to a healthy patient. Therefore, the systolic waveform 802 may demonstrate increased pressure relative to a patient having normal compliance, while the diastolic waveform 801 may demonstrate reduced pressure relative to a patient having normal compliance. Therefore, the resulting combined waveform 803 may represent an increase in the systolic peak and a drop in the diastolic pressure, which may cause various health complications. For example, the change in waveform may impact the workload on the left ventricle and may adversely affect coronary profusion.

In view of the health complications that may be associated with reduced arterial compliance, as described above, it may be desirable in certain patients and/or under certain conditions, to at least partially alter compliance properties of the aorta or other artery or blood vessel in order to improve cardiac and/or other organ health. Disclosed herein are various devices and methods for at least partially restoring compliance to a blood vessel, such as the aorta. Certain embodiments disclosed herein achieve restoration of arterial compliance through the use of implantable compliant fluid containers, which can be used to implement arterial bypass channels in some embodiments. For example, a compliance-restoration device in accordance with the present disclosure may comprise an expandable fluid container body portion/member that can be in/to an arterial wall, wherein the body portion/member is configured to be implanted at least partially within an adjacent vein. The device can include anchor structures that are configured to support/maintain port openings through the artery and vein walls to provide fluid communication between the artery and the compliant body portion disposed within the vein. The device may be anchored to the blood vessel wall using any suitable type of anchor means, such as a wire-form or stent anchor. Although certain embodiments of compliance restoration devices are described herein in the context of deployment in the aorta and inferior vena cava, it should be understood that compliance restoration devices in accordance with the present disclosure may be deployed in any chamber of the heart or any major artery or vein that may benefit from increased compliance characteristics. Compliance restoration devices disclosed herein may serve to at least partially increase coronary perfusion.

### Compliance-Restoration Implant Devices

The present disclosure relates to systems, devices, and methods for adding back compliance to the aorta and/or other arterial (and/or venous) blood vessel(s) to provide improved perfusion of the heart muscle and/or other organ(s) of the body. For example, embodiments of the present disclosure can include compliant tubular bypass devices configured to bypass flow from the aorta and/or other arterial blood vessel into the inferior vena cava and/or other venous blood vessel, such that aortic/arterial blood passes through a portion of the venous blood vessel(s) (e.g., inferior vena cava).

By bypassing arterial blood flow through a compliant fluid container disposed at least in part within a venous blood vessel, embodiments of the present disclosure can increase arterial compliance in a manner that presents a reduced risk of clotting/embolism formation compared to certain other compliance-restoration solutions. Furthermore, with the fluid container body disposed within a venous blood vessel, as opposed to external to the blood vessel(s), incidences of blood leakage and/or rupture of the container may be contained within the blood vessel(s), thereby reducing hazards associated with extravascular arterial blood leakage, such as within the abdominal and/or chest cavity. Rather, such blood leakage may be deposited within the venous system, resulting in little or no harm to the patient. Furthermore, devices disclosed herein can be implanted using a transcaval delivery/access, thereby allowing for delivery system components and/or other working instruments to be advanced through the venous system (e.g., inferior vena cava), rather than the arterial system, which can allow for relatively larger-profile devices/systems to be used and/or otherwise provide a relatively safer access and procedural implementation for implantation of the device(s).

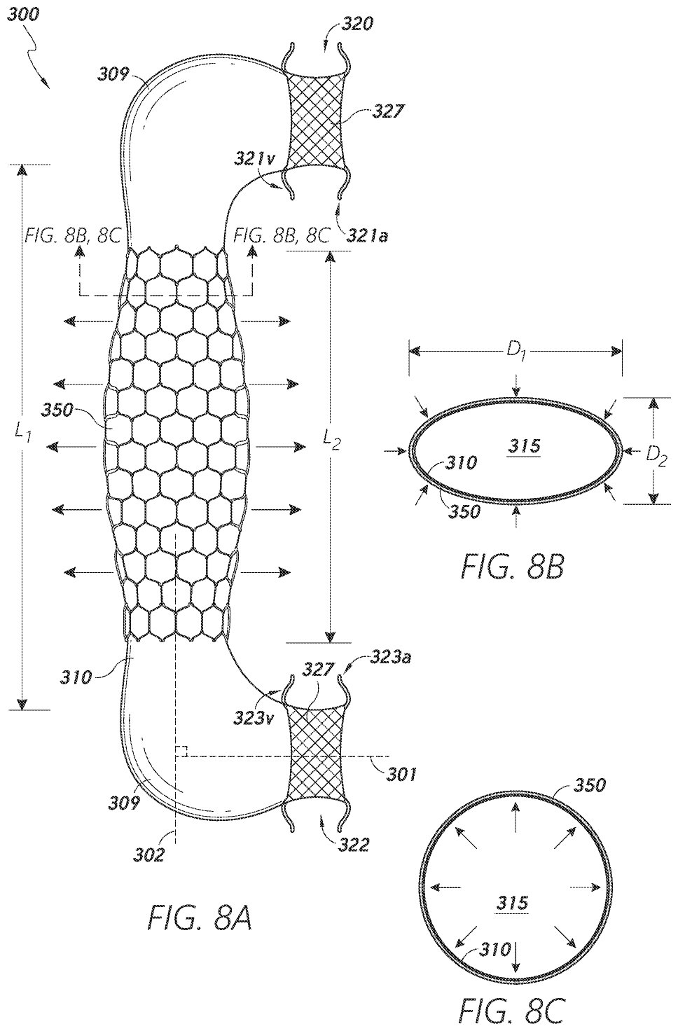

FIG. 6 is a cross-sectional view of a compliance-restoration device 100 implanted in arterial 15 and venous 19 blood vessels in accordance with one or more embodiments, the compliance-restoration implant device 100 is shown as implanted in a manner as to provide a blood flow bypass channel 115 into which blood may flow from the artery (e.g., aorta) 15 and back out into a downstream area of the artery 15. The bypass structure 110 is advantageously compliant. In FIG. 6, bypass structure 110 is shown as implanted in the inferior vena cava 19, wherein the compliance-restoration device 100 includes a first port structure 120 and a 2nd port structure 122 configured to provide fluid access from the aorta 15 into the elastic bypass structure 110 within the inferior vena cava 19. However, it should be understood that embodiments the present disclosure relate implant devices that may be implanted in any arterial and/or venous blood vessel(s).

The elastic bypass structure 110 may be a tubular bypass member. The compliant bypass structure 110 is advantageously compliant and configured to expand with respect to one or more dimensions during systole and store energy that is released when the bypass structure 110 contracts or otherwise deforms during diastole in response to changes in pressure in the arterial 15 and/or venous 19 blood vessels. The implant device 100, when implanted, resides at least partially inside the venous blood vessel 19 (e.g. inferior vena cava), where pressure may be generally lower compared to the arterial blood vessel 15 (e.g. aorta). Superior 120 and inferior 122 port structures can be included that are configured to facilitate a seal between the openings of the arterial wall 79 and the venous wall 78 and the implant device 100, which can allow blood from the arterial blood vessel 15 to flow into and back out of the compliant bypass structure 110 during systole and diastole, respectively. The port structures can be configured to maintain the opening(s) in the arterial 79 and venous 78 walls and may comprise certain wall anchor structure(s) (e.g., memory metal frames).

As shown in FIG. 6, the arterial (e.g., aortic) blood flow can pass through an opening 101 through the walls of the artery 15 and adjacent vein (e.g., inferior vena cava) 19, respectively. The term “opening” is used herein according to its broad and ordinary meaning. With respect to implant devices of the present disclosure as implanted in one or more blood vessels, the term “opening” may refer to an opening within an aortic blood vessel, a venous blood vessel, and/or the combination of an opening through both an arterial blood vessel wall and an at least partially over lapping opening in a venous blood vessel wall, such that the overlap of the openings provides a single opening through both blood vessel walls. The opening 101 may be maintained by the port/anchor structure 120, which may have any suitable or desirable structure or form, such as a stent, shunt and/or other structure.

Although illustrated with one or more port/anchor structures 120, 122, it should be understood that implant devices of the present disclosure may be implanted without including port/anchoring structures. For example, the compliant (e.g., stretchy, elastic) bypass structure 110 may be secured in place to provide one or more fluid ports/openings 101, 102 without a separate structural feature to hold such openings open and/or to secure the implant to the blood vessel wall(s).

The compliance-restoration device 100 includes a superior inlet port 120 and an inferior outlet port 122. The device 100 can be anchored in the arterial wall 79 and/or the wall 78 of the venous blood vessel 19 (e.g. inferior vena cava) 78 using any suitable or desirable anchoring means, such as one or more contact arms, flanges, grommets, sutures, tabs, hoops, wire forms, barbs, and/or the like.

The bypass blood flow from the arterial blood vessel 15, through the compliant bypass structure/body 110, and back into the arterial system, as indicated by the illustrated arrows in FIG. 6, can provide increased arterial compliance with reduced clotting risk due to the lack of blood pooling in the elastic bypass structure due to the directional flow therein. By placing the body of the implant device, referred to herein as the compliant bypass structure 110, in the venous blood vessel 19 (e.g. inferior vena cava), compliance can be added back to the arterial blood vessel 15 as well as to the venous blood vessel 19. For example, as pressure increases within the arterial blood vessel 15, increased flow may enter the bypass structure 110, thereby causing the compliant bypass structure to expand outwardly or otherwise deform due to the compliant and/or elastic characteristics thereof. As pressure in the arterial blood vessel 15 decreases, energy stored in the compliant bypass structure 110 due to expansion thereof can cause the bypass structure 110 to contract, thereby pushing blood flow out of the bypass structure 110 and back into the arterial system such that compliance is added back to the arterial system. Furthermore, expansion of the compliant bypass structure 110 within the venous blood vessel 19 can increase pressure in the venous blood vessel 19 and/or push fluid disposed therein in a manner as to increase compliance and/or flow within the venous system to some degree. Therefore, the single implant device 100 can serve to increase compliance in both the arterial and venous systems of the circulatory system of the patient.

By implanting the device 100 such that the compliant bypass structure 110 is disposed at least partially within the venous blood vessel 19, in the event that the implant device 100 leaks or ruptures in some manner, such leakage may be maintained substantially within the circulatory system, and particularly in the venous system (e.g., inferior vena cava 19). Such leakage within the venous blood vessel(s) may result in relatively less damage/injury to the patient compared to blood flow leakage outside of the circulatory system within the body cavity. For example, leakage within the venous blood vessel may result in substantially no damage or injury to the patient.

The bypass structure 110 can be constructed of a compliant material, such as an elastomeric polymer or other material. In some embodiments, the compliant bypass structure 110 comprises a woven structure, such as a woven memory metal braided structure, or the like. Furthermore, as the compliant bypass structure 110 is configured to be implanted within the venous blood vessel (e.g., inferior vena cava) 19 and/or in a plurality of venous blood vessels (e.g., inferior vena cava and iliac vein), the material from which the compliant bypass structure 110 is formed can be semipermeable in some implementations, as some amount of blood seepage through the flexible membrane of the compliant bypass structure 110 into the venous system may be acceptable/inconsequential and/or present reduced risk of adverse effects.

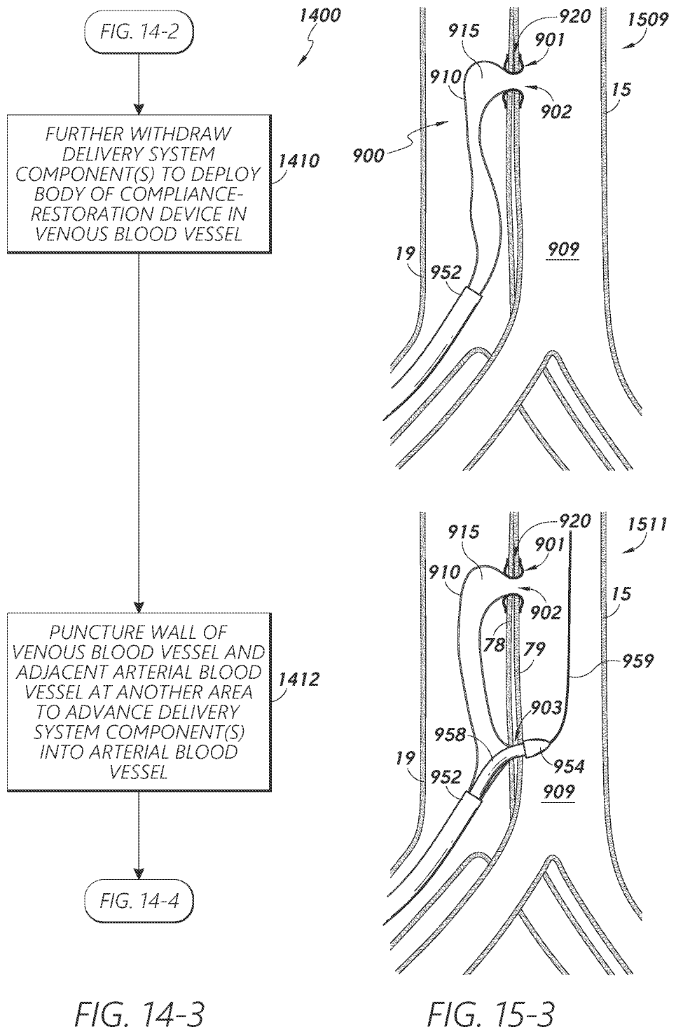

Although the compliant bypass structure 110 is illustrated as a tubular bypass structure, it should be understood that the compliant bypass structure 110 may have any suitable or desirable shape or form. For example, the bypass structure 110 may have a pouch-type form that may not necessarily be tubular in shape. Furthermore, although the structure 110 is described as a bypass structure, in some embodiments, as illustrated in FIG. 13 of the present disclosure and described in further detail below, the structure 110 may not provide by pass blood flow from an upstream port/opening in the arterial blood vessel 15 to a downstream port/opening in the arterial system, but rather may circulate blood into the structure 110 through a port/opening through the arterial wall, wherein blood is introduced back into the arterial system through the same port, such that substantially no segment of the arterial system is bypassed through the device 100.

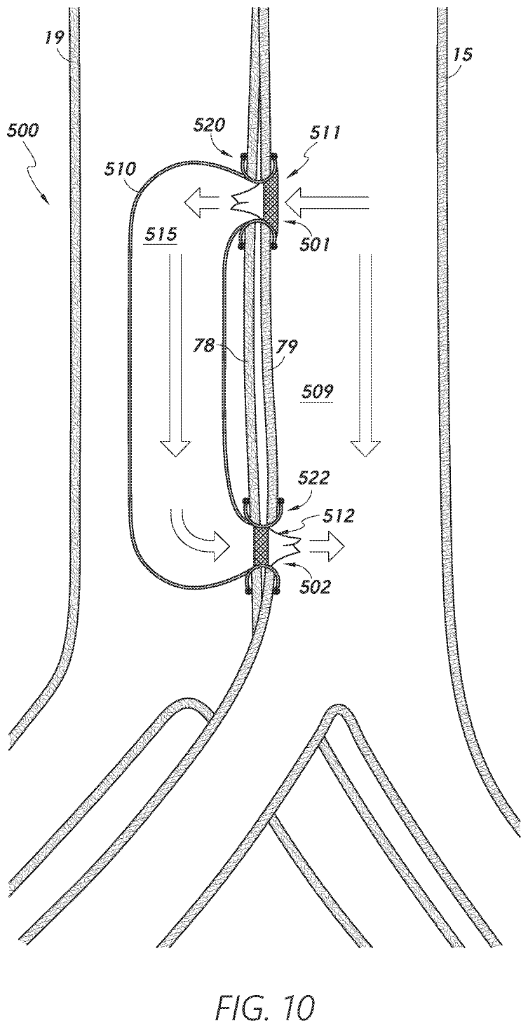

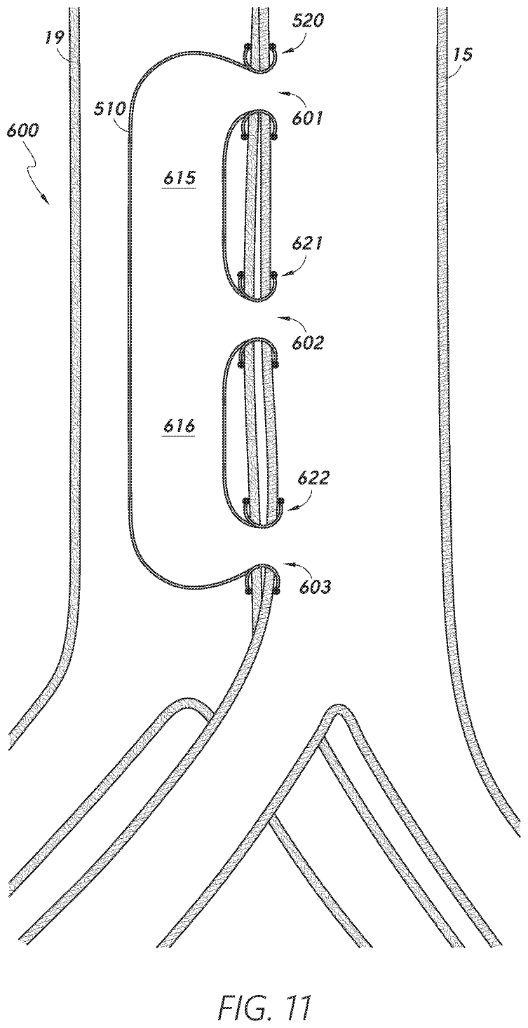

In some embodiments, the bypass structure 110 comprises biological tissue in addition to, or as an alternative to, a polymer or elastomeric material (see. e.g., FIGS. 8A-8C). For example, bovine pericardial tissue may be utilized to form the bypass structure 110, wherein a secondary structure, such as a memory metal braid or frame, may be secured around the structure 110 to allow the implant device 100 to expand/stretch and retract/recover, as necessary to reintroduce compliance to the system.

Generally, a pressure gradient may exist between the arterial blood vessel 15 and the venous blood vessel 19, wherein the pressure level within the arterial blood vessel 15 is greater than the pressure within the venous blood vessel 19 through at least a portion of the cardiac cycle. Furthermore, in some situations, a pressure level exterior to the blood vessels (e.g., within the abdominal and/or chest cavity) may be greater than that within the venous blood vessel 19. Therefore, blood shunted out of the arterial blood vessel 15 through the opening 101 in the vessel wall 79 may be inclined to enter into the venous blood vessel 19 through the opening in the venous vessel wall 78 rather than escape into the surrounding anatomical area outside of the vasculature. In view of such conditions, it may not be necessary for the anchor structures 120, 122 to provide complete fluid sealing between the blood vessels and/or around the openings therein.

Although the compliant bypass structure 110 is described as being at least partially permeable to blood in some embodiments, it may be advantageous for the bypass structure 110 to be substantially fluid-tight, such that blood cannot permeate the walls of the bypass structure 110. For example, such fluid tightness may facilitate the elastic expansion or other deformation of the structure in the presence of increased fluid pressure therein, which serves to increase the compliance-restoring characteristics of the implant device 100.

The elastic/compliant characteristics of the bypass structure 110 may advantageously increase compliance in a manner as may not be achievable without such elastic/compliant characteristics. For example, in the absence of elastic/compliant features, the bypass structure 110, without the ability to change in volume in response to increases in pressure therein, may simply serve to expand the total volume of the arterial system without absorbing and returning energy to/from the system and/or resulting in a change in volume of the vasculature throughout the cardiac cycle, which may generally not improve compliance.

The compliant bypass structure 110 may be sized and/or configured, such as with respect to a cross-sectional diameter thereof in one or more portions of the structure, such that the structure 110 does not occlude the venous blood vessel 19 in a disadvantageous manner. Alternatively, the structure 110 may be sized and/or dimensioned such that the structure 110 substantially occludes the venous structure 19 in one or more periods of the cardiac cycle. In some embodiments, the compliant bypass structure 110 is constructed in a manner as to limit expansion thereof in response to increasing pressure conditions, such that the structure 110 does not expand to degree to cause undesired occlusion of the venous blood vessel 19. For example, the expandability of the structure 110 may have a structural limit beyond which it will not expand further regardless of increases of pressure therein. The implant device 100 can span any suitable or desirable length L.

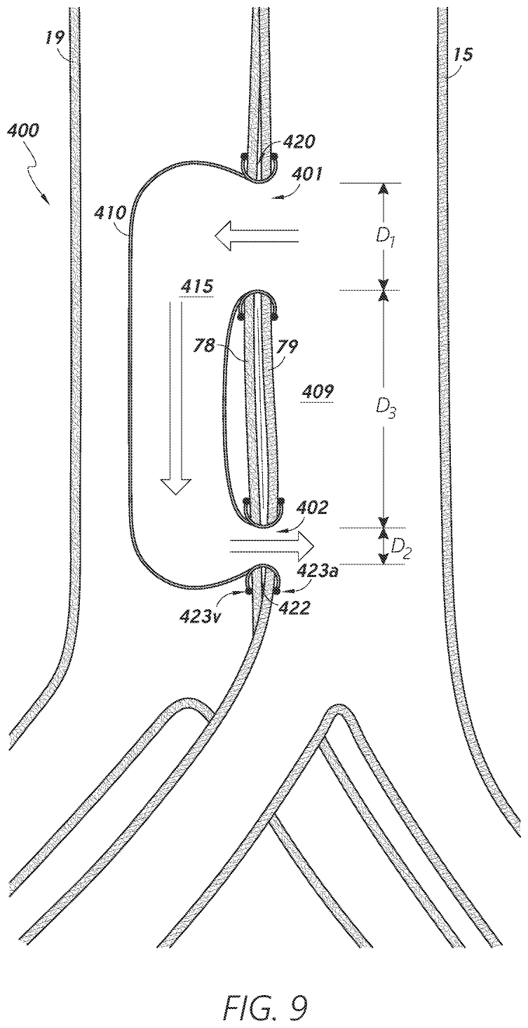

FIG. 7 shows a side view of a compliance-restoration device 200 including stent-type port reinforcement structures 220, 222 in accordance with one or more embodiments. The compliance-restoration implant device 200 may be similar in one or more respects to the compliance-restoration implant device 100 shown in FIG. 6 and described above. The implant device 200 may include an elastic/compliant tubular bypass structure 210, which may be in fluid communication with inlet 201 and outlet 202 openings therein, which serve as bypass flow ports as described in detail herein.

The inlet 201 and outlet 202 ports can be reinforced with respective stent frames 227, which may form at least part of respective port/anchor structures 220, 222. For example, the stent frames 227 can comprise self-expanding memory metal frames that are configured to expand to form a suitable fluid seal within blood vessel wall openings, as described herein. Furthermore, the frames 227 can serve to approximate the arterial 79 and venous 78 walls when implanted as shown in FIG. 3 and described above. For example, when implanted, the anchor structures 221, 223 can hold the walls 79, 78 together in some manner to cause such blood vessel walls to be approximated to one another, thereby reducing the risk of fluid leakage outside of the vasculature.

The reinforcement stents 227 can have any suitable or desirable length L. The length L can be dimensioned to span a distance between the interior of the target arterial blood vessel (e.g., aorta) and an interior of the adjacent target venous blood vessel (e.g., inferior vena cava). For example, in some implementations, the stent structures 227 may have a length L of approximately 1-3 cm. Within such range, relatively wider lengths L may be implemented for relatively heavily-calcified aortas/blood vessels.

FIG. 8A shows a side view of a compliance-restoration device 300 including a compliant sleeve 350 associated with a body portion 310 thereof in accordance with one or more embodiments. FIGS. 8B and 8C show cross-sectional views of the compliant sleeve 350 of FIG. 8A in compressed and expanded configurations, respectively, in accordance with one or more embodiments. It should be noted that although FIGS. 8B and 8C shows cross sectional area change in the D₁ and D₂ directions, in some embodiments, diameter change in bypass conduit sleeves can be substantially uniform rather than predominantly in one direction/dimension.

Although various embodiments disclosed herein including compliant fluid container structures, wherein increases in pressure result in stretching/expansion of such structures, it should be understood that in some embodiments, such compliance may be achieved through the use of one or more secondary structures associated with the implant device. For example, tubular bypass structures disclosed herein may be compliant in some embodiments, such that the material of the bypass structure provides compliance characteristics for the implant device. However, in some embodiments, such tubular bypass structures may not comprise elastic and/or compliant material, but rather compliance may be provided to the implant device through the disposition of a compliant reinforcement structure. For example, the compliance-restoration device 300 shown in FIGS. 8A-8C includes a tubular bypass structure 310 that has a reinforcing compliance member 350 associated therewith. For example, the compliance member 350 may comprise a cylindrical/tubular structure disposed about the tubular bypass structure 310, wherein the compliance structure 350 is configured to expand outwardly with respect to one or more dimensions thereof in response to radial forces thereon from within the tubular structure 310.

According to some examples, the bypass structure 310 may comprise biological tissue, such as bovine pericardium, or a polyester material that is not elastic in nature. However, elasticity/compliance can be provided by the implant device through the placement of the compliance sleeve 350 over the tubular structure and/or incorporated in some manner with the body/tube 310 of the implant device 300, as shown in FIGS. 8A-8C.

The sleeve member 350 can be configured to expand and contract with each cardiac cycle, thereby storing energy and returning such energy to the circulation system in a manner as to increase compliance thereof. The sleeve 350 can comprise a flexible material, such as a memory metal frame or the like, a woven elastic fabric/sleeve, and/or electroactive material. That is, the sleeve 350 may improve compliance of the device 300 by stretching and expanding radially in response to pressure increases and returning to a non-stretched, or less stretched, condition as pressure decreases, thereby returning energy to the fluid disposed therein. In some embodiments, the sleeve is shape biased to a non-circular shape (e.g., oval), wherein such divergence from a circular cross-sectional shape can provide room for cross-sectional area expansion as the cross-sectional area becomes more circular in response to pressure increases without the need for elasticity in the walls of the bypass structure 310. This manipulation of the blood vessel walls can introduce more volumetric change in response to the typical changes in pressure experienced during the cardiac cycle, thereby increasing cardiac efficiency and reducing the pulsatile load.

As referenced above, in some embodiments, the sleeve 350 does not stretch in an elastic manner, but rather may provide compliance through a reshaping of the cross-sectional area thereof, as demonstrated in FIGS. 8B and 8C. For example, in FIG. 8B, the sleeve 350 may comprise memory metal that is biased to oval shape having a first dimension D₁ that is greater than a second dimension D₂. As shown in FIG. 8C, in the presence of radial outward force from within the sleeve 350, the sleeve 350 may assume a more circular cross-sectional shape, as shown. Generally, as understood by those having ordinary skill in the art, the area of the circular configuration shown in FIG. 8C, assuming substantially no change in perimeter/circumference length, may be generally greater than the area of the oval configuration shown in FIG. 8B, and therefore as pressure is reduced and the sleeve 350 is biased back toward the oval configuration of FIG. 8B, energy may be introduced back into the circulatory system to improve/increase compliance thereof. That is, whether through elastic stretching or cross-sectional reshaping, the sleeve 350 advantageously is configured to expand and recover as a function of pressure conditions therein such that the internal volume of the sleeve 350 changes throughout the cardiac cycle, thereby introducing compliance to the system.

With further reference to FIG. 8B, the diastolic flow within the arterial system can be is enabled by the decrease in cross-sectional area of the bypass channel 310 during diastole, which forces blood through the channel and back into the artery. That is, the differential cross-sectional area of the bypass structure 310 between the systolic and diastolic phases facilitates compliance and perfusion. Generally, for a stiff aorta, the cross-sectional area of the aorta may not change from systole to diastole, and therefore cardiac perfusion suffers. The sleeve 250 can cause a change in the cross-sectional shape of the bypass structure 310, which acts as a surrogate for the arterial blood vessel, to a non-circular shape (e.g. oval, racetrack, triangular, etc.). The device 300 therefore leverages the principle that an ellipse or other non-circular cross-sectional shape will have lesser area than a circular cross-sectional shape having the same perimeter. In some embodiments, the sleeve 350 is further configured to stretch in addition to, or as an alternative to, cross-sectional reshaping, which may provide improved compliance restoration characteristics compared to solutions involving a fluid container body portion that either stretches, or is shape-biased to a non-circular cross-sectional shape, but not both.

In some embodiments, the sleeve 350 can have an opened configuration prior to coupling with the device 300. That is, the sleeve 350 may have features that allow for the sleeve to be placed around the bypass structure 310 of the device 300. For example, a connection means may be used to secure the sleeve 350 around the bypass structure 310. The connection means may allow for coupling of the sleeve 350 after implantation of the device 300. Any type of connection means may be used, including latches, magnets, snaps, and the like. The connection means may comprise a hinging feature, a suture, a separate attached component, or other mechanism, in some implementations, the sleeve may be dimensioned to be slid over the body 310 of the device prior to implantation of the device 300.

The sleeve 350 may have any suitable or desirable length L₂. For example, the length L₂ may represent a portion of the length L₁ between the ports 320, 322, as implanted. For example, in some embodiments, the implant device 300 may include support structures 320, 322 configured to be implanted in a first axial orientation 301, wherein, as implanted, the tubular bypass structure 310 may be oriented at a substantially perpendicular orientation 302 over at least a portion of a length thereof that corresponds to the length L₂ of the sleeve 350. For example, the tubular bypass structure 310 may include one or more bends 309 allowing for the blood flow to flow into and out of the implant device 300 in the first orientation 301, and flow through the body of the tubular bypass structure 310 along the orthogonal/perpendicular orientation 302, which may be substantially parallel to an axis of the bypassed arterial blood vessel.

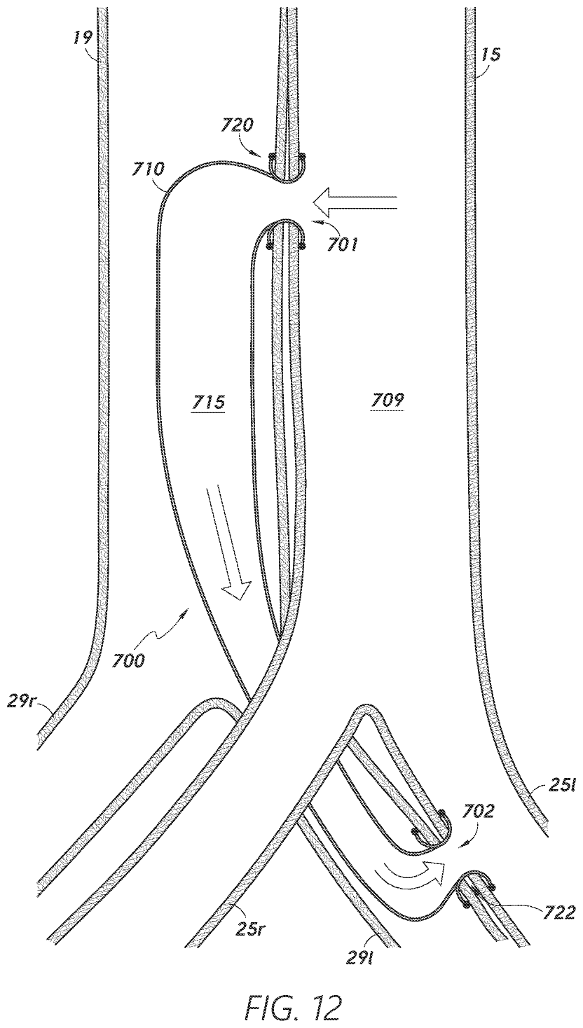

FIG. 9 is a cross-sectional view of a compliance-restoration device 400 including port structures 420, 422 having differing geometries in accordance with one or more embodiments. According to some examples, embodiments of the present disclosure may advantageously facilitate directional bypass flow from an inlet port of a compliance-restoration device to an outlet port thereof. For example, where blood flows through an arterial blood vessel in a given direction, it may be desirable for bypass fluid routed from such blood vessel to flow in a common direction with the bypassed blood vessel. Embodiments of the present disclosure may include certain flow-controlled characteristics to insure and/or promote flow in such direction.

Flow control for compliance-restoration implant devices in accordance with aspects of the present disclosure may be achieved through the use of port structures having certain absolute and/or relative sizes. For example, an upstream inlet port structure 420 may be configured with a flow channel area having a diameter or other dimension D₁ that is greater than a corresponding diameter/dimension D₂ associated with a downstream support structure 422. For example, in some embodiments, an inlet port may have a diameter of approximately 2-3 cm, whereas an outlet port may have a diameter of approximately 1-2 cm. With a relatively enlarged inlet port structure 420, the pressure associated with inflow into the channel 415 of the device 400 may be relatively lower compared to the outflow from the downstream support structure 422, thereby promoting directional flow from the inlet port structure 420 to the outlet port structure 422. Therefore, substantially parallel flow streams may flow in the bypassed segment 409 of the arterial blood vessel 15 as within the bypass channel 415 of the compliance-restoration device 400. That is, the bypassed flow in the channel 415 may be implemented to mirror the natural direction of blood flow through the arterial blood vessel 15.

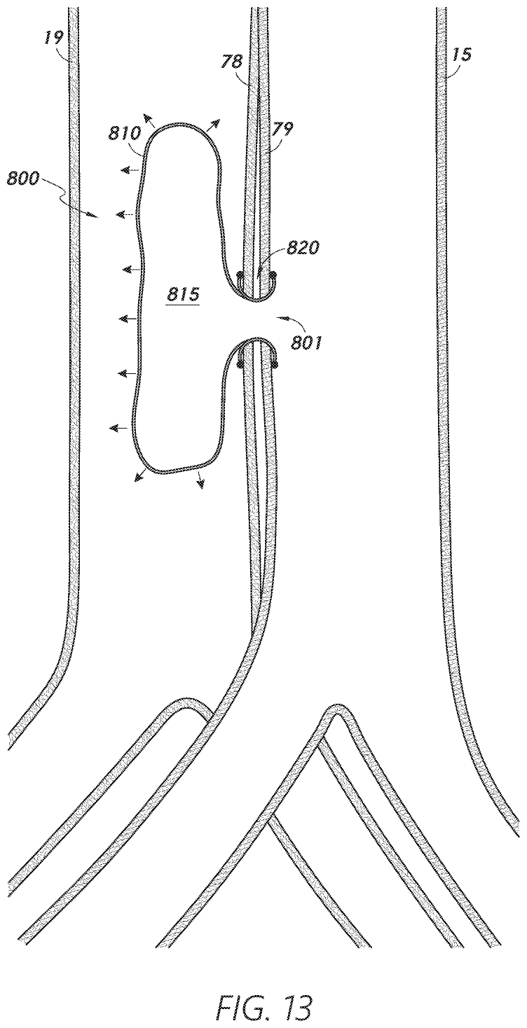

FIG. 10 is a cross-sectional view of a compliance-restoration device 500 including flow-control features in accordance with one or more embodiments. In addition to, or as an alternative to, utilizing differently-sized inlet and outlet ports to provide flow-control functionality for a compliance-restoration bypass implant device in accordance with aspects of the present disclosure, various other flow-control mechanisms may be utilized to achieve the desired direction and/or volume or rate of flow through a bypass implant device in accordance with aspects of the present disclosure. For example, valves or other flow-control features can be associated with one or more portions of a compliance-restoration implant device, such as at or near one or more ports of the implant device. The compliance-restoration implant device 500 shown in FIG. 10 includes one or more one-way valves 511, 512, which may be configured and/or oriented to permit flow in a desired direction through the channel 515 of the bypass structure 510, while restricting or blocking flow in the reverse direction.

In some embodiments, a compliance-restoration implant device in accordance with aspects of the present disclosure may include a one-way valve associated with an inlet port structure 520 of the implant device 500. For example, the port structure 520 may include a stent frame or other structure configured to maintain an opening in the tissue wall 79, 78 and/or for anchoring the implant device 500 to the tissue wall(s) 79, 78. An interior of such frame may have associated therewith and/or secured thereto a one-way valve 511, as shown in FIG. 10. In some implementations, a second one-valve 512 may further be implemented. For example, in embodiments including a plurality of one-way flow-control valves, one such valve may be coupled to and/or associated with an inlet port structure 520, whereas another may be coupled to and/or otherwise associated with an outlet port structure 522, as shown in FIG. 10. The valve features associated with compliance-restoration devices in accordance with aspects of the present disclosure may function by opening to permit flow in the presence of a pressure gradient in the direction of the valve, such that the valves open to permit flow and close with each cardiac cycle.

Although FIG. 10 shows two one-way valves 511, 512 associated with respective ports of the compliance-restoration device 500, it should be understood that compliance-restoration devices in accordance with aspects of the present disclosure may include any number of valves, wherein such valve(s) may be positioned, disposed, and/or configured at any suitable or desirable position of the implant device 500. For example, in some embodiments, one-way valve feature(s) may be associated with the bypass tube/structure 510.

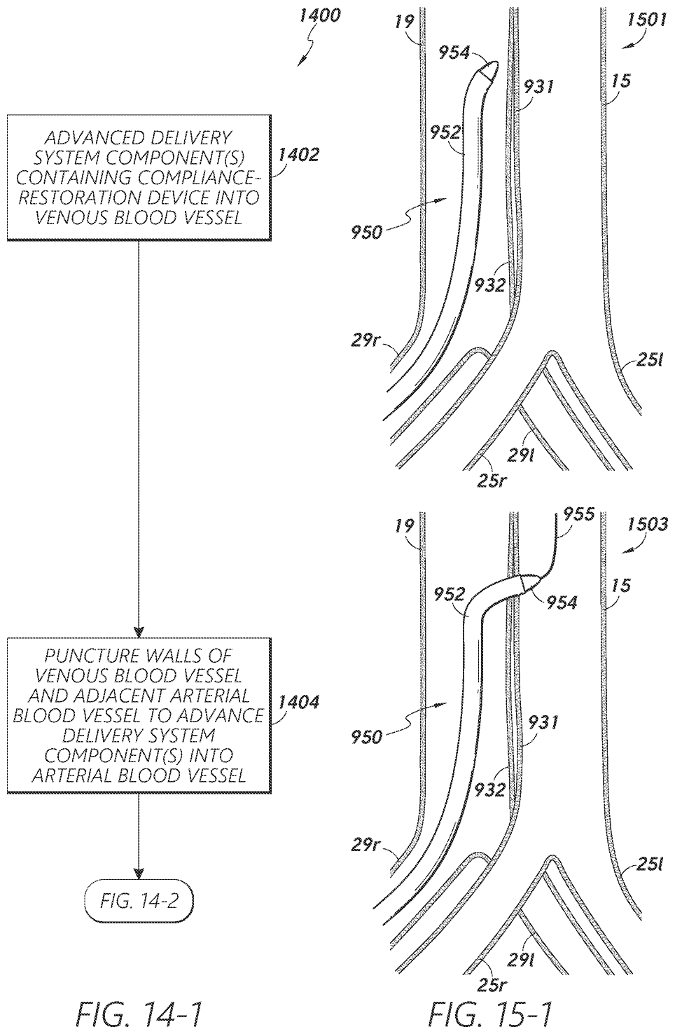

FIG. 11 is a cross-sectional view of a compliance-restoration device 600 including more than two ports in accordance with one or more embodiments. Certain compliance-restoration implant devices are illustrated and disclosed herein as comprising two fluid access ports, namely a single fluid inlet port and a single fluid outlet port. However, it should be understood that compliance-restoration devices in accordance with aspects of the present disclosure may include any suitable or desirable number, arrangement, size, and/or configuration of ports.

FIG. 11 shows a compliance-restoration implant device 600 including more than 2 ports 601-603. Depending on the relative sizes of the port structures 620, 621, 622 associated with the respective ports, in operation, each of the ports may serve primarily as either an inlet port or an outlet port. The use of more than two ports may allow for a desired amount of bypass flow with relatively smaller port sizes. That is, the total amount of port area for fluid flow may be achieved using a greater number of ports as opposed to a lesser number of larger ports. The use of relatively smaller port sizes may be advantageous in view of the curvature of the target blood vessels and/or anatomy in which the implant device is implanted. For example, it may be desirable for a port to have a dimension that is relatively small with respect to the circumference of the blood vessels in which it is anchored to thereby reduce the amount of reshaping of the blood vessels when implanting the compliance-restoration device. Furthermore, relatively smaller fluid inlet/outlet ports may allow for smaller puncture holes in the blood vessels, thereby potentially reducing the risk of injury or damage to the patient's blood vessels. In some embodiments, it may be desirable for fluid inlet and/or outlet ports of a compliance-restoration device in accordance with aspects of the present disclosure to have a diameter that is approximately 1.5 cm, or less.

Depending on where the implant device 600 is implanted, the arterial blood vessel 15 in the area of implantation may be associated with certain arterial branches, which may service the liver, kidneys, stomach, and/or other organ(s). For example, while the abdominal area of the aorta 15 shown in FIG. 11 may be generally free of arterial branches (and venous branches with respect to the inferior vena cava 19 adjacent to the abdominal aorta), areas farther up the vascular anatomy may include vascular off-shoots, and therefore it may be desirable to reduce the profile/footprint of the respective ports of the implant device 600 to allow for greater flexibility and placement around or near blood vessel branches.

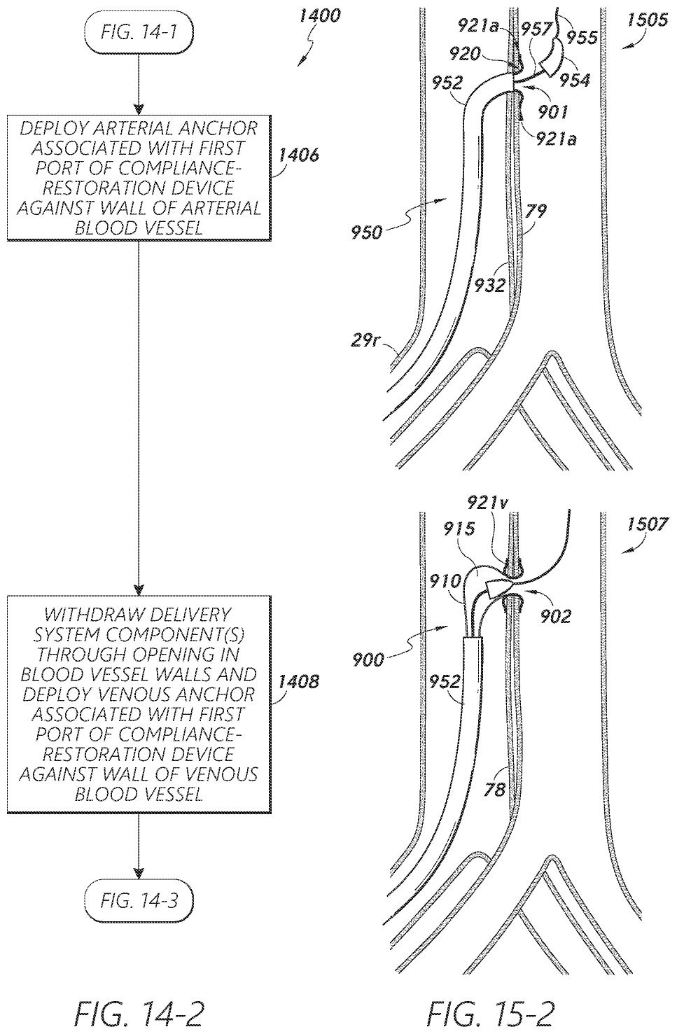

FIG. 12 is a cross-sectional view of a compliance-restoration device 700 implanted in arterial and venous blood vessels in accordance with one or more embodiments. Although certain embodiments are disclosed herein and described as being implanted in a manner as to bypass fluid from one portion of the aorta to another portion of the aorta, it should be understood that devices of the present disclosure may be configured to be implanted in any arterial and/or venous blood vessels. Furthermore, in some implementations, one port of a compliance-restoration implant device may be implanted in the aorta and inferior vena cava, whereas another port may be implanted in one or more other blood vessels in fluid communication therewith. For example, with respect to the compliance-restoration device 700, the bypass ports 720 and 722 may traverse not just the aorta 15 and inferior vena cava 19, but also other large conduit vessels, such as the iliac arteries and/or veins.

As shown in FIG. 12, a compliance-restoration implant device in accordance with aspects of the present disclosure may be implanted with respect to one or more ports thereof in an iliac artery 25 and/or vein 25. For example, in the illustrated embodiment of FIG. 12, the implant device 700 includes a port structure 722 implanted in the walls of the left iliac artery 25l and the left iliac vein 291. It should be appreciated that the port structure 722 may be implanted in the right iliac artery 25r and/or right iliac vein 29r. In the illustrated example of FIG. 12, blood flow through the channel 715 of the implant bypass structure 710 can bypass a segment 709 of the abdominal aorta as well as a portion of the iliac artery 25 and deposit blood channeled through the channel 715 into the iliac artery 25l.

In some contexts, the iliac arteries and veins may be considered parts of the same blood vessels as the aorta and inferior vena cava, respectively. That is, references to a blood vessel in which a compliance-restoration implant device in accordance with aspects of the present disclosure is implanted may refer to fluidly coupled trunks and branches of a blood vessel system/tree. Therefore, where a reference is made to a compliance-restoration device in which separate port structures thereof are described as anchored to and/or implanted in a particular blood vessel, it should be understood that such implant devices may be implanted in different branches or in a trunk and branch of a common blood vessel system/tree. Alternatively, branches of a blood vessel system/tree may be referred to and considered as separate blood vessels relative to the trunks from which they emanate in some contexts for clarity.

FIG. 13 is a cross-sectional view of a single-port compliance-restoration device 800 implanted in arterial and venous blood vessels in accordance with one or more embodiments. In some embodiments, compliance-restoration implant devices in accordance with aspects of the present disclosure do not include separate inlet and outlet ports, but rather a single port that provides access to a compliance chamber that can expand within a venous blood vessel, such as the inferior vena cava, in response to increases in pressure in the arterial blood vessel (e.g., aorta) in which the port structure is implanted/anchored. For example, a single port structure 820, as shown in FIG. 13, can allow for the implant device 800 to serve the function of a compliant chamber, rather than a bypass channel, which may be desirable in certain instances.

The pouch structure 810 of the compliance-restoration implant device 800 may comprise a balloon, flexible sack, and/or the like, wherein a body portion thereof is configured to be disposed within the venous blood vessel 19 when the anchor/port structure 820 is anchored in a manner as to provide a port 801 through the walls and 79, 78 of the arterial and venous blood vessels, respectively. It may be desirable for the pouch 810 to be significantly compliant/elastic in order to provide for relatively forceful ejection of blood therefrom into the arterial blood vessel 15 in order to reduce the risk of blood stagnation/pooling within the pouch 810, which can present embolization risks. Although shown as a flexible pouch, the compliance chamber 810 can include a stent/sleeve as described above with respect to FIGS. 8A-8C. For example, the pouch 810 may comprise a memory metal frame or woven mesh, which may facilitate expansion and return as described in detail herein. In some embodiments, the pouch 810 may advantageously comprise memory metal or other material that is less prone to breakage or degradation over time compared to certain polymeric materials and structures.

### Compliance-Restoration Device Implantation Processes

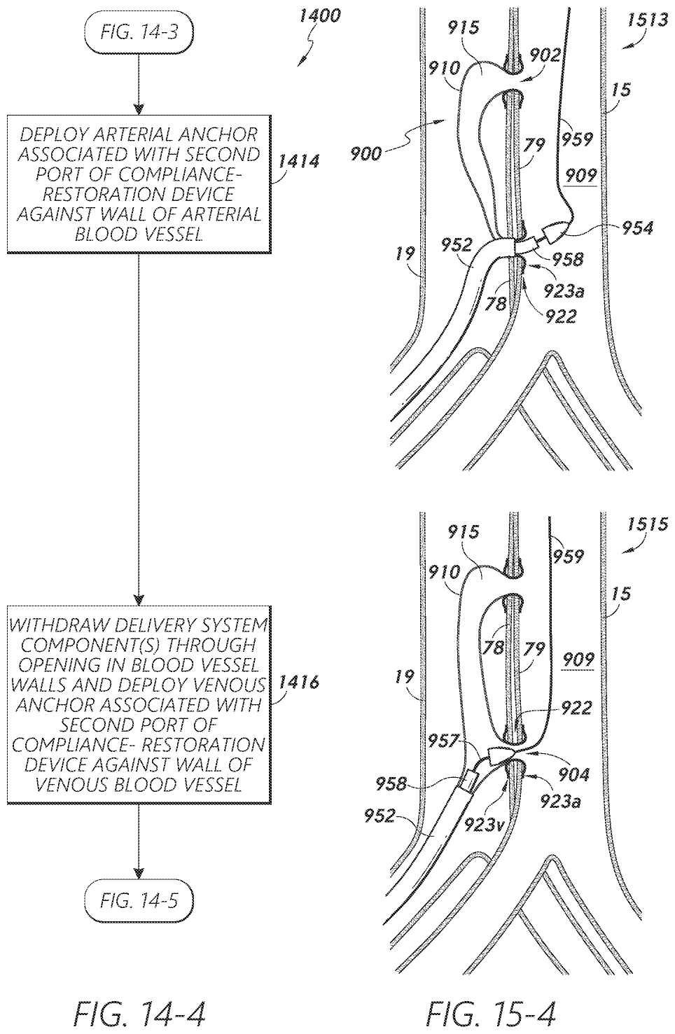

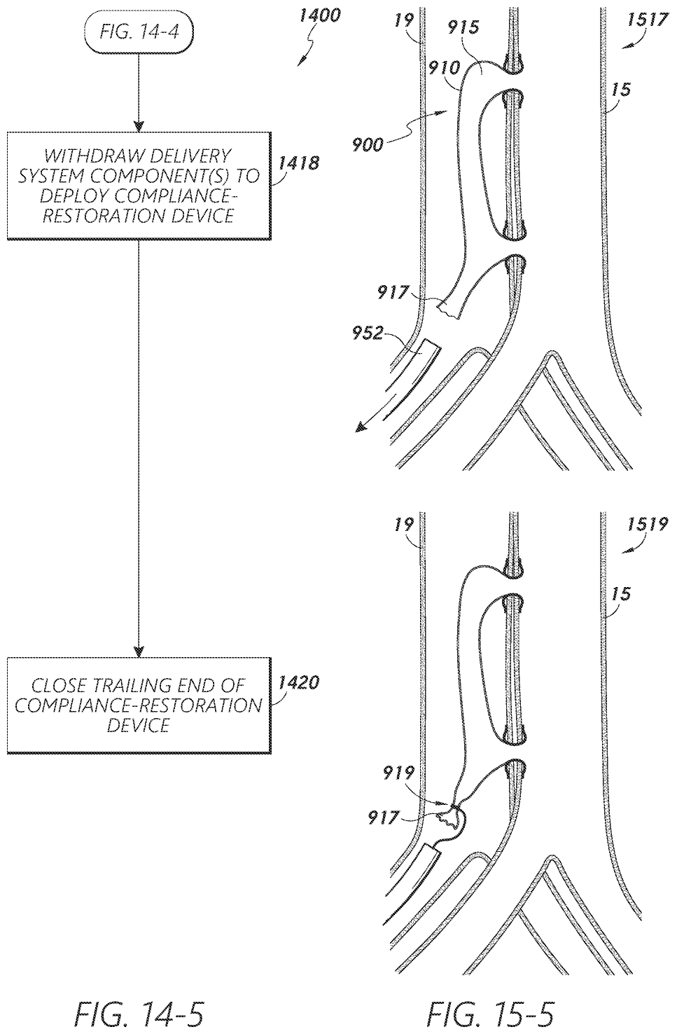

FIGS. 14-1, 14-2, 14-3, 14-4, and 14-5 illustrate a flow diagram for a process for implanting a compliance restoration device in accordance with one or more embodiments. FIGS. 15-1, 15-2, 15-3, 15-4, and 15-5 provides images of the compliance restoration device and certain anatomy corresponding to operations of the process of FIGS. 14-1, 14-2, 14-3, 14-4, and 14-5 according to one or more embodiments.

At block 1402, the process 1400 involves advancing one or more delivery system components containing a compliance restoration device into a venous blood vessel 19, such as the inferior vena cava, via one of the iliac veins 29. For example, the compliance-restoration device may be contained within a delivery catheter in a crimped or otherwise compressed configuration to allow for transportation thereof transvascularly. For example, a guidewire may be introduced into the femoral vein and further into the inferior vena cava through a percutaneous access.

At block 1404, the process 1400 involves puncturing the walls of the inferior vena cava 19 or other venous blood vessel and adjacent arterial blood vessel (e.g. aorta 15) to advance one or more of the delivery system components into the arterial blood vessel 15. For example, for implantation of a compliance-restoration device in accordance with aspects of the present disclosure in the abdominal space of a patient, a transcaval procedure may be implemented, wherein access to the aorta is made via the inferior vena cava by puncturing the blood vessel walls separating the arterial and venous blood vessels and advancing the delivery system through the opening formed therein. Transcaval procedures may be preferable when implanting devices disclosed herein for patients presenting anatomical conditions in which the arterial system is difficult to access and/or navigate within. For example, relatively small, tortuous, and/or heavily calcified aortas can be better suited for transcaval access. Furthermore, pressure conditions in the arterial system may make it difficult or untenable to access the aorta via the femoral artery or other arterial access.

In some implementations, fluoroscopy or other imaging technology may be used to assist in puncturing from the inferior vena cava into the adjacent aorta, wherein such puncture may be made either mechanically or electrosurgically. A sheath device can be advanced over the wire 955 to dilate through the walls of the inferior vena cava 19 and aorta 15 using a dilator tip 954, as shown in image 1504. When puncturing through the blood vessel walls as shown in image 1504, the pressure in the abdominal cavity may generally be higher than the fluid pressure in the venous blood vessel 19, such that any blood leakage from the arterial blood vessel 15 may be inclined to enter into the vein 19 rather than leaking indiscriminately into the abdominal cavity, which may facilitate implementation of a transcaval procedure without undue risk of injury.

At block 1406, the process 1400 involves deploying one or more arterial vessel anchor features or means associated with a first port or port structure of the compliance-restoration device 900 against and/or to the wall 79 of the arterial blood vessel 15, as shown in image 1506. For example, once the delivery system has crossed over into the arterial blood vessel 15, the arterial anchor(s) 921a may be deployed from the catheter/sheath 1952, wherein such anchor feature(s) may serve to retain the implant device in a manner as to resist the device being pulled back through the opening 1901 in the blood vessel walls. For example, the arterial anchor means/features may comprise one or more hooks, barbs, flanges, arms, clamps, tabs, sutures, and/or the like. Deployment of the anchor(s) 921a may be achieved at least in part by pulling back the sheath 952 to expose the anchors 921a, wherein the port/anchor structure 920 and/or anchoring means 921a may be configured to expand when released from the sheath 1952. For example, the anchor(s) 921a may be configured with shape-memory characteristics that cause the features to expand to assume an anchoring configuration when deployed from the sheath 1952.

In some embodiments, the anchor(s) 921a is/are configured to be attached to the arterial wall 79 in some manner and/or embedded therein, or may simply serve to present a diameter for the port structure 920 of the implant device that is greater than the opening 901 such as to prevent the device from being pulled back through the opening 1901. Over time, tissue ingrowth may secure the anchor(s) 921a to the arterial wall 79.

At block 1408, the process 1400 involves withdrawing the delivery system component(s) through the opening 1901 in the blood vessel walls and deploying one or more venous anchors 921v associated with the port structure 920 of the compliance restoration device against the wall 78 of the venous blood vessel 19. For example, the implant device 900 may be further unsheathed from the sheath 952 to expose and/or deploy the venous anchor(s) 921v.

In some embodiments and/or implementations, the compliance-restoration implant device 900 does not include the venous anchor(s) 921v. That is, the arterial anchor(s) 921a may be sufficient to secure the implant in place to the blood vessel wall(s). In some embodiments, the arterial anchor(s) 921a and venous anchor(s) 921v may be configured to close together to pinch the blood vessel walls therebetween and secure the port structure 920 in place. Such an implementation may cause the anchors to heal together and form a relatively secure fluid seal around the opening 901. In some embodiments, the anchors comprise a grommet-type attachment mechanism. In some embodiments, the anchor features may be injectable, wherein some aspect thereof can be inflated with an adhesive that can be cured to form a fluid seal around the port structure 920, opening 901, and/or anchors 921.

At block 1410, the process 1400 involves further withdrawing the delivery system component(s) to deploy the body portion 910 of the compliance restoration device 900 at least partially within the venous blood vessel (e.g., inferior vena cava) 19. For example, the body portion 910 may comprise a tubular bypass structure/conduit, which may advantageously be elastic and/or compliant, as described in detail herein.

At block 1412, the process 1400 involves puncturing the walls of the venous blood vessel 19 and adjacent arterial blood vessel 15 at another location to advance the delivery system component(s) into the arterial blood vessel 15 via a secondary opening 903 in the blood vessel walls. For example, the opening 903 can be downstream of the opening 901 with respect to the artery 15. For example, implementation of the operations associated with block 1412 may involve, once the sheath 952 has been withdrawn to the area of the secondary puncture/opening 903, withdrawing the nosecone 954 towards and/or into the sheath 952 and advancing a guide wire 959 through the puncture opening 903 via a mechanical or electrosurgical puncture, thereby producing a relatively small port through which the nosecone 954 may be advanced to dilate the opening 903.

At block 1414, the process 1400 involves deploying arterial anchor(s) 923a associated with a second port structure 922 of the compliance-restoration device 900 against the wall 79 of the artery 15. At block 1416, the process 1400 involves withdrawing the delivery system catheter/sheath 952 through the opening 903 in the blood vessel walls and deploying venous anchor(s) 923v, against the wall 78 of the venous blood vessel 19. The port anchor(s) 923v can help to create a leak-resistant port from the aorta/artery 15 through the vein 19 (e.g., inferior vena cava) into the body 910 of the implant device 900. Although the flow diagram 1400 describes only two port structures of the implant device 900 being implanted in the blood vessel walls, it should be understood that the process 1400 may involve the implantation of any number of port structures and anchoring thereof to the respective blood vessel walls. That is, the operations associated with blocks 1412-1416 can be repeated as needed to install any number of additional ports.

At block 1418, the process 1400 involves withdrawing the delivery system catheter/sheath 952 to fully deploy the compliance-restoration implant device 900. In some implementations, deployment of the implant device 900 may involve or require inclusion of an open-ended tail portion 917 to allow for removal of one or more delivery system components therethrough to complete deployment. At block 1420, the process 1400 involves closing or sealing-off in some manner the tail portion 917, such as through the use of one or more sutures 919, clips, clamps, or other sealing means or tools. In some embodiments, the tail portion 917 may be configured to automatically close using shape memory features or other components biased to a closed configuration to at least partially block fluid flow through the tail portion 917 in the implanted configuration. Other scaling means may be utilized, including certain belts, straps, clamps, or the like, which may be configured to automatically engage on the tail portion 917, or be secured to the tail portion 917 using appropriate working instruments. In some embodiments, the suture(s) or other sealing means 919 can be pre-attached to the tail portion 917, such that it is not necessary to reengage the tail portion 917 to seal it off after the implant 900 has been deployed as shown in image 1517.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment. Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of shunting blood, the method comprising:
forming a first opening in a wall of a first blood vessel and a wall of a second blood vessel;
anchoring a first port of a compliant fluid container to the wall of the first blood vessel such that the first port provides access between the first blood vessel and the second blood vessel through the first opening; and
placing a body of the compliant fluid container within the second blood vessel.

2. The method of claim 1, wherein:
the first blood vessel is an artery; and
the second blood vessel is a vein.

3. The method of claim 1, further comprising channeling blood from the first blood vessel into the body of the compliant fluid container within the second blood vessel through the first port.

4. The method of claim 3, further comprising:
forming a second opening in the wall of the first blood vessel and the wall of the second blood vessel;
anchoring a second port of the compliant fluid container to the wall of the first blood vessel such that the second port provides access between the first blood vessel and the second blood vessel through the second opening; and
channeling blood from the body of the compliant fluid container into the first blood vessel through the second port.

5. The method of claim 4, further comprising passing blood through the body of the compliant fluid container between the first port and the second port.

6. The method of claim 4, wherein the first port is upstream of the second port with respect to blood flow within the first blood vessel.

7. The method of claim 6, further comprising:
forming a third opening in the wall of the first blood vessel and the wall of the second blood vessel;
anchoring a third port of the compliant fluid container to the wall of the first blood vessel such that the third port provides access between the first blood vessel and the second blood vessel through the second opening; and
channeling blood between the first blood vessel and the body of the compliant fluid container through the third port.

8. The method of claim 3, further comprising:
forming a second opening in a wall of a third blood vessel and a wall of a fourth blood vessel;
anchoring a second port of the compliant fluid container to the wall of the third blood vessel such that the second port provides access between the third blood vessel and the second blood vessel through the second opening; and
channeling blood from the body of the compliant fluid container into the third blood vessel through the second port.

9. The method of claim 8, wherein a portion of the body of the compliant fluid container is disposed within the fourth blood vessel.

10. The method of claim 9, wherein:
the first blood vessel is an aorta;
the second blood vessel is inferior vena cava;
the third blood vessel is an iliac artery; and
the fourth blood vessel is an iliac vein.

11. The method of claim 1, wherein the first port is formed by an anchoring structure of the compliant fluid container that is disposed within the first opening.

12. The method of claim 11, wherein the anchoring structure comprises a stent configured to hold open the first opening.

13. The method of claim 1, further comprising adding compliance to the first blood vessel by filling the body of the compliant fluid container with blood from the first blood vessel to thereby expand the body of the compliant fluid container within the second blood vessel.

14. A compliance restoration implant device comprising:
a compliant fluid container configured such that a cross-sectional area of the fluid container increases when a pressure level within the fluid container is greater than a pressure level outside of the fluid container and decreases when the pressure level within the fluid container is less than the pressure level outside of the fluid container, and
a first port structure coupled to the fluid container and configured to provide fluid access to an interior of the fluid container.

15. The compliance restoration implant device of claim 14, wherein the first port structure is configured to be anchored to a blood vessel wall.

16. The compliance restoration implant device of claim 15, wherein the first port structure comprises a stent frame.

17. The compliance restoration implant device of claim 16, further comprising a second port structure coupled to the fluid container and configured to provide fluid access to the interior of the fluid container.

18. The compliance restoration implant device of claim 17, wherein:
the first port structure is coupled to a first end of the fluid container; and
the second port structure is coupled to a second end of the fluid container.

19. The compliance restoration implant device of claim 18, further comprising a third port structure coupled to the fluid container and configured to provide fluid access to the interior of the fluid container.

20. The compliance restoration implant device of claim 19, wherein the first port structure has an opening that is greater than an opening of the second port structure.

21. The compliance restoration implant device of claim 20, wherein the fluid container comprises:
a tubular member; and
a sleeve disposed about the tubular member.

22. The compliance restoration implant device of claim 21, wherein the sleeve is configured such that a cross-section thereof changes from an oval shape to a more circular shape in response to an increase in pressure within the tubular member.

23. The compliance restoration implant device of claim 22, wherein the sleeve is elastic.

24. The compliance restoration implant device of claim 23, wherein the sleeve comprises a memory metal frame.

25. The compliance restoration implant device of claim 24, wherein the sleeve comprises a braided mesh.

26. A fluid bypass implant device comprising:
a compliant tubular structure;
a first fluid port associated with a first end of the tubular structure; and
a second fluid port associated with a second end of the tubular structure.

27. The fluid bypass implant device of claim 26, wherein each of the first fluid port and the second fluid port comprises an anchoring means configured to anchor to an interior wall of a blood vessel.

28. The fluid bypass implant device of claim 27, wherein the anchoring means comprises one or more anchoring arms that extend from a respective one of the first fluid port and the second fluid port and are configured to contact the interior wall of the blood vessel.

29. The fluid bypass implant device of claim 28, wherein the anchoring means comprises a flange structure.

30. The fluid bypass implant device of claim 26, further comprising a flow control means disposed at least partially within a fluid channel of the fluid bypass implant device.

31. The fluid bypass implant device of claim 30, wherein the flow control means comprises a one-way valve.

32. The fluid bypass implant device of claim 26, further comprising one or more valve devices coupled respectively to one or more of the first fluid port and the second fluid port.
